(19) United States
(12) Patent Application Publication
(10) Pub. No.: US 2019/0240083 A1
(43) Pub. Date: Aug. 8, 2019

(54) LIQUID ABSORPTION AND DISTRIBUTION NONWOVEN FABRIC FOR HYGIENE ARTICLES

(71) Applicant: Sandler AG, Schwarzenbach (DE)

(72) Inventors: Uwe Bernhuber, Hof (DE); Martin Schuberth, Stammbach (DE); Marco Damke, Selb (DE); Andreas Burger, Selb (DE)

(21) Appl. No.: 16/261,728

(22) Filed: Jan. 30, 2019

(30) Foreign Application Priority Data
Feb. 2, 2018 (DE) 10 2018 000 854.2

(51) Int. Cl. A61F 13/513; D04H 3/14; D04H 1/559; A61F 13/511

(52) U.S. Cl. CPC A61F 13/513; D04H 3/14; A61F 2013/51016; A61F 13/51121; D10B 2509/026; D04H 1/559

(57) ABSTRACT

This disclosure describes a multi-layer liquid absorption and distribution nonwoven fabric for hygiene products, which has a topside facing the user in hygiene products and an underside facing the absorbent core in hygiene products, the fiber blend of the nonwoven fabric forming the underside having a lower average fiber titer than the fiber blend of the nonwoven fabric forming the topside. The bond between the topside and the underside is purely thermal. The topside consists of a fiber blend of 50 to 100% by weight of melting fibers with 50 to 0% by weight of matrix fibers. The underside consists of a blend of 50 to 80% by weight of absorbent fibers with 50 to 20% by weight of melting fibers.

### CROSS-REFERENCE TO RELATED APPLICATIONS

This application claims priority to German Patent Application No. DE10 2018 000 854.2, filed on Feb. 2, 2018, the disclosures of which are incorporated herein by reference in its entirety.

### FIELD OF THE INVENTION

This disclosure relates to multi-layer liquid absorption and distribution nonwoven fabrics for hygiene products.

### BACKGROUND OF THE INVENTION

Disposable articles for hygiene applications, such as sanitary towels, diapers, and incontinence products are designed to wick body liquids away from the skin as quickly as possible to prevent skin irritation and give the user a safe and comfortable feeling. For this purpose, hygiene products are generally structured as follows: first layer: topsheet (perforated sheet or nonwoven fabric); second layer: acquisition and distribution layer, “ADL” (mostly made of nonwoven fabric); third layer: absorbent core (consisting of cellulose and/or polyacrylic acid fixed in one or multiple fixing layers (e.g., SMS); and final layer: sheet for preventing leakage of liquid on the rear side.

The topsheet, ADL and absorption core layers must be matched to each other to guarantee the function of the hygiene product. The most important role of ADL is to serve as a transfer layer. This has to absorb the liquid of the topsheet (drainage of the top sheet), distribute the liquid in the longitudinal direction of the product and intermediately store the liquid for a short period. The intermediate storage is intended to prevent the so-called gel blocking of the polyacrylic acid powder. After the brief intermediate storage, the liquid is transferred to the absorption core for final storage.

To achieve this objective, acquisition and distribution layers (abbreviated as “ADL” from “Acquisition Distribution Layer”) made of non-woven fabrics are used to perform the following functions: fast absorption and transfer of the liquid; intermediate storage for buffering the limited absorption speed of the absorption material; directional distribution of the liquid for the largest possible absorption by the absorber material; and controlled release of the liquid to the absorber material to prevent local blockage and liquid over-accumulation.

Thus, the most important properties of an absorption and distribution nonwoven fabric are thickness, density, and the resulting pore sizes as well as capillarity. These properties have a decisive influence on the absorption and distribution behavior. In this respect, air laid, high loft and spunlace nonwoven fabrics are used in some products. For example, the application DE 10 2012 015 219 A1 describes various types of spunlace nonwovens with corresponding properties.

The types of nonwoven fabrics described in the state of the art are mainly interlinings with homogeneously distributed fiber types. Depending on the manufacturing method, only one required property can be achieved at a time. Therefore, for example, highloft nonwoven fabrics, i.e., nonwoven fabrics purely thermally bonded by means of melting fibers, offer a high density due to their structure and thus a good liquid absorption. However, due to the comparatively high porosity, the distribution effect is very low. Thus, only a very limited use of the underlying absorbent core is possible.

In Contrast, the nonwoven fabrics manufactured with the airlaid process have a good distributive effect due to the fine capillaries. Due to the high cellulose content, however, the thickness is low, and the liquid is also released back to the surface, which results in a high rewet.

Two-layer structures resulting from a one or two-stage manufacturing process, such as in DE102016005158, are likewise not expedient. The layers are bonded using water jets, whereby, for instance, hydrophilic agents are washed away, such that liquid absorption is insufficient. Furthermore, the resulting final thickness is insufficient due to the type of solidification, as the ADL side, in particular, can only absorb a small amount of liquid.

A bonding of the layers in multilayer structures by means of an adhesive, such as hotmelt via spray coating or slot coating, results in sufficient layer adhesion, but the hotmelt makes liquid transfer more difficult. Areas blocked with hotmelt, which are impermeable to liquids, are formed at the transitions of the layers.

State-of-the-art materials combine the properties of liquid distribution and liquid absorption whereby smears in the respective effect are desirable. Therefore, there remains a strong need for a material which avoids the aforementioned disadvantages of the state of the art and has an improved liquid absorption at the simultaneously given liquid distribution.

### SUMMARY OF THE INVENTION

This disclosure provides a multilayer liquid absorption and distribution nonwoven fabric for hygiene products. The nonwoven fabric comprises a topside facing a user in hygiene products and an underside facing the absorbent body in hygiene products. A fiber blend of the nonwoven fabric forming the underside has a lower average fiber titer than a fiber blend of the nonwoven fabric forming the topside. In some embodiments, the topside consists of a non-woven fabric and the fiber blend of the topside consists of 50 to 100% by weight of melting fibers and 50 to 0% by weight of matrix fibers. In some embodiments, the underside consists of a non-woven fabric, and the fiber blend of the underside consists of 50 to 80% by weight of absorbent fibers and from 50 to 20% by weight of melting fibers. In some embodiments, the topside is exclusively thermally fusion-bonded to the underside, and the density of the nonwoven fabric is 35 kg/m³ or less.

In some embodiments, an average fiber titer of the fiber blend of the nonwoven fabric forming the underside is at least 2 dtex less than an average fiber titer of the fiber blend of the nonwoven fabric forming the topside. In some embodiments, the topside is thermally fusion-bonded to the underside using a hot-air treatment or using a hot-air treatment and a downstream calender treatment. In some embodiments, the thickness of the topside is at least 60% of the total thickness. In some embodiments, the fusible components of the melting fibers in the topside and in the underside are made of the same polymers. In some embodiments, the density of the underside is at least twice as high as the density of the topside.

### DETAILED DESCRIPTION OF THE INVENTION

This disclosure addresses the need in the art by providing a nonwoven fabric with its vertical structure (i.e., perpendicular to the plane of the layers), the topside, or the underside manufactured with different fiber compositions and, if necessary, also different types of bonding. In addition, each side can be designed in such a way that the respective function is optimally fulfilled by the fiber blend and/or the type of bonding.

In some embodiments, the layers may be bonded exclusively thermally, with the fiber blend on the topside containing more than 50% by weight of melting fibers. This ensures that a sufficient number of bonding points are formed between the topside and the underside.

The transition of the zones also creates a gradient of physical properties, such that the topside has the greatest permeability and mechanical stability, while the underside facing the absorption core has the greatest capillarity. Such gradient transports the liquid quickly and almost completely away from the body side and forced by physical forces to the absorption core.

Some of the terms used in the following description are defined in more detail below:

Textile fibers have so-called “avivages,” which are finishing agents, on the fiber surface in order to improve the processing properties and guarantee certain effects in the finished product.

An “absorption fiber,” in the sense of the present invention, is a regenerated cellulose fiber produced from solutions of cellulose derivatives. This viscose fiber can have a surface modification, e.g., trilobal cross-section, but can also be modified in such a way that a moderate absorption of liquid is favored with good liquid storage at the same time. Representatives, according to the invention, include, for example, Viscostar viscose fibers or normal viscose fibers from the manufacturer Lenzing, as well as so-called Galaxy fibers from the company Kehlheim Fibers. In particular, viscose fibers produced according to the Lyocell process (Tencel by Lenzing) can also be used. The usual fiber finenesses range from 1.0 to 3.3 dtex, preferably 1.3 to 2.2 dtex. If staple fibers are used, the fiber lengths used range from 10-70 mm, preferably between 35-50 mm. The absorption fibers have commercially standard crimps. However, the term “absorption fiber” can also refer to short fibers or pulp if the underside is an airlaid.

The term “matrix fiber” is used for staple fibers of thermoplastic and/or thermosetting polymers contained in the topside. Fibers made of thermoplastic polymers, such as polyester or polypropylene, are preferred. The melting temperature must be at least 10° C. above the melting fibers used. Thermosetting polymers, such as polyacrylic fibers, are also suitable. The fiber finenesses range from 3.3 to 17 dtex, preferably 4.4 to 10.0 dtex; the fiber lengths used are in the range of 10-80 mm, preferably 35-60 mm. The distribution fibers have commercially standard 2D or 3D crimps.

The terms “lubricating” or “avivage” describes a functional finish/coating on fiber surfaces. These avivages are commonly used as processing aids, i.e., fibers are treated in such a way that static charging is prevented, particularly with PP or PE fibers. Avivages are usually applied to the surfaces of staple fibers. The type of avivage selected can influence hydrophilicity or hydrophobicity, which can have a major influence on the properties of the liquid absorption and distribution nonwoven fabric. The avivages are present on the surfaces of the melting fibers as well as matrix fibers and absorbent fibers.

The term “melting fibers,” as used herein, refers to fibers which are contained in a product constructed according to the invention to enable the bonding of the topside with the underside fiber composite and to stabilize the structure of the topside, such that a largely dimensionally stable nonwoven fabric is produced.

Melting fibers are homo- or bi-component, thermoplastic polymer fibers that have fusible components. When exposed to temperature, these components melt and stabilize the fiber composite after cooling. Fibers suitable, according to the invention, are homopolymeric staple fibers of co-polyester, co-polyamide or polypropylene, bicomponent melting fibers in core-sheath or side-by-side arrangement of combinations of low-melting co-polyester with polyester, polyethylene with polypropylene or polyethylene with polyester are preferred, according to the invention. The fiber finenesses for the melting fibers used in the topside are in the range of 2.2 to 12 dtex, preferably 4.4 to 6.7 dtex; the fiber lengths used are in the range of 10-80 mm, preferably 35-60 mm.

The fiber finenesses for the melting fibers used in the underside are in the range of 1.7 to 3.3 dtex, preferably 1.7 to 2.2 dtex; when staple fibers are used, the fiber lengths used are in the range of 10-80 mm, preferably 35-60 mm; when short fibers are used, should the underside be formed by an airlaid nonwoven fabric, the range is between 1 and 5 mm, preferably 3 mm. If the melting fibers are cardable staple fibers, they will have commercially standard crimps.

Nonwoven fabrics, according to the invention, are manufactured using dry or wet processes; the underlying processes can be found in the book “Vliesstoffe” (English: “Nonwoven Fabrics”), published in 2000 by Wiley VCH-Verlag, Weinheim. In particular, the nonwoven fabric forming the underside can be an airlaid nonwoven fabric, a so-called “airlaid.” According to the invention, carded staple fiber nonwoven fabrics are preferred.

The fibers used are homogeneously blended with each other when manufacturing a fiber blend. The basic techniques are also described in the book “Vliesstoffe.”

According to the invention, the “topside” is the side, which is turned towards the user in the hygiene product, and on which the liquid first comes into contact. The “underside” is the side of the nonwoven fabric according to the invention, which is arranged facing the absorbent core in the hygiene product.

The term “precursor” refers to a prefabricated nonwoven fabric, which has already undergone the process steps of fiber blending, nonwoven fabric formation, and nonwoven fabric bonding. According to the invention, the underside is formed by a precursor.

The following examples refer to, but are not limited to, staple fiber nonwoven fabrics manufactured according to a dry process using the carding process.

TABLE 1 lists the test results obtained in each case. The parameters determined were determined according to the following test methods:
Basis weight per unit area according to WSP 130.1., specified in kg/m²;
Thickness according to WSP 120.6, section 7.2, measuring pressure 0.5 kPa, specified in m;
Bond strength according to NWSP 401.0.R0(15), specified in N;
The thickness of the topside is determined using the following formula:

Topside thickness(m)=total thickness(m)−precursor thickness(m)

Density is determined according to the following formula (1):

$$Density ( kg \text{/} {m}^{3} ) = \frac{Basis weight per unit area ( kg \text{/} {m}^{2} )}{Dry thickness ( m )} \qquad ( 1 )$$

Strike-through according to WSP 70.3, specified in s;

Rewet according to WSP 80.10, specified in g;

Average fiber titer within one layer: calculated according to the following formula (2):

$$Average fiber titer ( dtex ) = \frac{A * Titer 1 + B * Titer 2 + C * Titer 3}{100} \qquad ( 2 )$$

A, B, C=percentage of a fiber component in the blend, wherein the sum of A, B, and C is 100;

Titer 1, 2, 3=Nominal titer of the respective fiber component in dtex.

The required properties, i.e., fast absorption of liquid, good distribution with short-term, intermediate storage and transfer to the absorption core, are achieved, according to the invention, by a structure consisting of two interbonded nonwoven fabric layers.

A liquid absorption and distribution nonwoven fabric manufactured according to the invention goes through the following process steps:
(a) Manufacturing a fiber blend for the precursor, consisting of absorbent fibers blended with melting fibers
(b) Manufacturing an unbonded material web from the fiber blend of the precursor.
(c) Thermal and/or mechanical bonding of the material web, such that the precursor is formed with a density of 50 to 200 kg/m³ and a thickness of 0.4 to 1.0 mm.
(d) Optional: Winding the precursor.
(e) Manufacturing a fiber blend for the topside, consisting of matrix fiber and melting fiber.
(f) Manufacturing an unbonded web material from the fiber blend of the topside.
(g) Laying the unbonded material web on the precursor.
(h) Solidifying the topside and bonding the topside to the precursor by means of a hot-air treatment.
(i) Optional: Additional passage through a calender to stabilize the bond. The bonding area should be less than 10%, preferably less than 7%.
(j) Winding of the liquid absorption and distribution nonwoven fabric, according to the invention.

The nonwoven fabric forming the underside consists, according to the invention, of a precursor having a content of 50 to 100% by mass of absorbent fiber. In the precursor, fiber blends are used which show a difference between the average fiber titer of the precursor and the topside. According to the invention, the difference must be at least 2 dtex, whereby the average fiber titer of the precursor fiber blend must be lower than that of the topside.

The average fiber titer of the precursor fiber blend is in the range of 1.0 to 6.0 dtex, preferably less than 4 dtex and most preferably lesser than 2.0 dtex.

If the precursor is manufactured as a carded staple fiber nonwoven fabric, as is preferred by the invention, this results in a fiber orientation in the production direction of the nonwoven fabric. This orientation is advantageous for later use, since the fiber orientation then also runs in the longitudinal direction of the finished hygiene product. This improves the utilization of the available absorption surface in the hygiene product.

When bonding of the precursor, which is preferred according to the invention, but not limited to, using water jets, the precursor is simultaneously compressed, such that the density of the precursor is higher than that of the final absorption and distribution nonwoven fabric. Densities of the precursor of 50 to 200 kg/m³, preferably 80 to 120 kg/m³, are envisaged, whereby the thickness range of the precursor lies between 0.4 and 1 mm and a basis weight per unit area of 0.025 to 0.120 kg/m² is intended.

Due to the selected average fiber titer of the precursor in connection with the compression during the bonding of the precursor, the capillary action and thus the capacity to transport liquid within the precursor is positively influenced.

According to the invention, an unbonded carded fibrous web is now placed on this precursor. After a subsequent solidification and bonding step, this fibrous web forms the topside of the nonwoven fabric according to the invention.

The solidification of the fibrous web of the topside and the bonding of the topside with the precursor is performed, according to the invention, by means of thermal treatment, i.e., by hot-air bonding and an optional subsequent calender bonding. The basic techniques can be found in the book “Vliesstoffe,” published by Wiley-VCH in 2012, pages 375-395.

The fibrous web of the topside consists of 50 to 0% by weight of matrix fibers and 50 to 100% by weight of melting fibers.

According to the invention, a proportion of more than 50% of melting fibers is necessary in the fiber blend that forms the topside. Compared to the state of the art, this proportion is significant and ensures a sufficient number of possible bonding points between the topside and the underside.

If the unbonded fibrous web, which forms the topside after bonding, is placed on the underside, there are only a few contact points at the boundary layer between the topside and the underside, which can fuse after hot-air treatment or form bonding points.

What is necessary and preferred according to the invention, but without being limited thereto, is therefore to provide a system for hot-air bonding that operates according to the throughflow principle. Hot air is directed onto the initially unbonded fibrous web of the topside and flows through the topside followed by the underside. Depending on the amount of hot air flowing through and the throughflow velocity, the fibrous web of the topside experiences a lower or higher densification in comparison to the thickness of the unbonded fibrous web.

By means of this densification, a significantly larger number of contact points and therefore possible bonding points of melting fibers are formed on the boundary layer topside to underside compared to the simple “laying on” of the fibrous web and solidification by means of jetting or radiant heat.

In order to guarantee the adhesion of the topside with the underside, melting fibers are used in the fiber blend of the underside. This is because the bonding between melting fibers is facilitated, and such bonding points of melting fibers from the topside to the lower side are mechanically more resistant.

Hot-air solidification and bonding has proven to be particularly advantageous with regard to the lubricating of the fibers of the topside. If a bond is produced by hydroentanglement, the avivage present on the fibers is almost completely washed away by the water jets. The fibrous or nonwoven fabric properties achieved by means of the avivage are thereby altered, such that requirement profiles cannot be met. The hot-air solidification and bonding process preserves the avivages on the fiber surfaces and does not affect the properties.

For the precursor, the upper limit for the content of melting fibers is 50% by weight, since with proportions greater than 50% by weight, the liquid storage suffers, and on the other hand, the precursor becomes too stiff.

According to the invention, identical melting fibers, i.e., made of the same polymers, can also be used, such that the adhesion of the topside to the underside is improved.

In another preferred embodiment, in particular for the case that the precursor contains only absorbent fibers, a melting fiber type with a particular affinity for cellulosic polymers can be used.

The average titer of the fiber mixture of the topside is greater than 3.3 dtex, preferably in the range from 4.4 to 12 dtex, and particularly preferred in the range from 6.7 to 12 dtex. Due to this average fiber titer, the nonwoven fabric forming the topside is open enough to absorb liquid quickly and store it temporarily until it is passed on to the underside. The higher the average fiber diameter of the blend, the better the resistance of the topside to pressure loads. As the proportion of melting fibers increases, the topside also becomes mechanically more stable, i.e., has improved resistance to compression, such as high packing density of diapers or to point loads during use. The rewet is positively influenced by higher average fiber titers and a melting fiber content of more than 75% by weight.

The thermal activation of the melting fibers guarantees, in particular, the stability of the topside. This ensures improved resistance to the aforementioned compression in the product application.

According to the invention, the bonding conditions in combination with the fiber blend of the topside are selected in such a way that the liquid absorption and distribution nonwoven fabric according to the invention has a thickness greater than 3 mm, preferably greater than 5 mm.

In the event that the percentage of melting fiber in the fiber blend forming the underside is less than 30% by weight, an optional thermal calender treatment can be provided to improve adhesion of the topside to the underside. The adhesion is determined by the bond strength.

In order not to influence the thickness of the liquid absorption and distribution nonwoven fabric, the arrangement of the calender and the calender engraving must, therefore, be chosen accordingly.

The gravure roller must be arranged in such a way that the embossing occurs on the topside. The gravure roller must have a pressing area of less than 10%, preferably less than 7%, on the one hand, and the web height, i.e., the distance from the engraving base to the pressing plateau, must be greater than 2 mm, on the other hand. According to the invention, engravings are used which, if not designed as lines, have less than 10 engraving points per cm², particularly preferred are engravings with 7 or less engraving points per cm².

The liquid absorption and distribution nonwoven fabric according to the invention has an open, mechanically stable nonwoven fabric structure to ensure liquid absorption in the topside and a densified underside for distribution to absorb liquids and distribute them due to capillarity.

The requirements on the topside are achieved by: (1) the average titer of the fiber blend >4.0 dtex; (2) the thickness of the topside >0.002 m; and (3) the weight of the topside >0.04 kg/m².

The requirements on the underside are achieved by: (1) the average titer of the fiber blend <6.0 dtex; (2) the thickness of the underside <0.001 m; and (3) the weight of the underside <0.06 kg/m².

The requirements for the bond of the topside and the underside, whereby the thickness of the topside is not significantly influenced, are achieved by the provision of: (1) the percentage of the melting fibers >50% by weight in the fiber blend of the topside; (2) 20% to 50% by weight of melting fibers in the fiber blend of the underside; (3) the application of the hot-air treatment with airflow from the topside through the underside; and (4) optional application of a subsequent calender treatment after the hot-air treatment.

The combination of features of the topside and the underside results in a liquid absorption and distribution nonwoven fabric designed according to the invention having the following features: (1) The thickness of the topside accounts for at least 60% of the total thickness; (2) The density of the underside is at least twice as high as the density of the topside; (3) The average fiber titer of the topside has to be at least 2.0 dtex greater than the average fiber titer of the underside; and (4) The density of the liquid absorption and distribution nonwoven fabric is a maximum of 35 kg/m³.

Referring to TABLE 1, the following can be observed:

Sample 1 represents a state-of-the-art liquid absorption and distribution nonwoven manufactured in accordance with DE102016005158. Water jets are used to bond the topside with the underside. Although there is an average fiber titer of 6.2 dtex of the topside and an average fiber titer of 2.2 dtex of the underside, the thickness is 0.00178 m due to the compression of the water jets. The strike-through is 9.4 s.

Samples 2 to 5 were treated by hot-air bonding in a belt dryer. For the underside, a nonwoven fabric already solidified with water jets was used as the precursor. The unbonded fibrous web was then, according to the invention, laid on this nonwoven fabric forming the underside and the fibrous web was solidified, so as to form the topside. Bonding the topside to the underside was achieved by directing the hot air from the topside towards the underside by means of a directed airflow. The dryer temperatures used depend on the type of polymer and melt viscosity. For samples 2 to 5, temperatures in the range of 120 to 140° C. were used, without being limited thereto.

Sample 2, according to the invention, was manufactured with identical fiber blends of the topside, as shown in Sample 1, and where the bonding between the topside and the underside was achieved by means of hot air on a flat belt dryer, according to the invention, it can be seen that the total thickness is higher by a factor of 3 compared to Sample 1. The lower compression of the topside and the presence of hydrophilic avivage on all fibers of the topside result in an improved strike-through compared to the state of the art. The bond strength is at an acceptable level, i.e., the bonding of the topside to the underside is maintained during processing. The difference between the average fiber titer of topside and underside is 4.5 dtex. Sample 1 has the same titer difference, but the topside has a much smaller thickness compared to the topside of Sample 2.

By using matrix fibers with very high titers, i.e., 10 dtex, the high thickness of the topside of Sample 2 is ensured. This ensures a very high porosity. The density of Sample 2 according to the invention is 17 kg/m³ which is 3.8 lower than that of Sample 1.

Sample 3, manufactured according to the invention, does not use matrix fibers. The percentage of melting fibers is 100% by weight. The difference of 3.1 dtex in the average fiber titer of the topside blend and the underside blend again results in the porosity of the topside. The bond strength is at a higher level compared to Sample 2 due to the melting fiber content of 100% by weight. The difference in the average fiber titer of the topside and the underside of Sample 3 is 3.1 dtex. The strike-through and rewet are in a range corresponding to Sample 2. The density is 29 kg/m³, which is almost twice as high as compared to Sample 2. This higher density causes a slightly higher intake.

Compared to Samples 2 and 3, Sample 4 provides for the use of only 20% by weight of melting fibers in the underside. The layer adhesion is therefore significantly lower compared to Samples 2 and 3, such that this proportion of melting fibers in the underside defines the lower limit.

In Sample 5, a calender treatment following hot-air bonding was carried out. A calender engraving was used, which has a pressing area of 5% and 9 figures/cm² with a web height of 2 mm. The pressure was 75 daN/cm, the roller temperature 125° C. The gravure roller pressed on the topside.

In addition to a positive influence on the layer adhesion, there is also a slight increase in the capillary effect on the surface of the topside. This favors a directed liquid transfer.

In Sample 5, where the average fiber titer of the topside is 4.4 dtex, the density is higher compared to Samples 2 to 4, but the strike-through and rewet are in the range of Samples 2 to 4.

In other versions according to the invention, calender engravings in the form of lines similar to DE10103627 can also be used. It should also be noted here that the web height is 2 mm and higher, and that the pressing area is less than 10%.

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

TABLE 1

| | | | | | Total | Average | Basis weight | | Strike- | |
|---|---|---|---|---|---|---|---|---|---|---|
| | | | Thickness | | Density | Fiber Titer | per unit area | | through | Rewet |
| | Layers | Blend | [m] | | [kg/m³] | [dtex] | [kg/m²] | | [sec] | [g] |
| Sample 1 | Topside | 30% Matrix fiber PET | 0.0013 | Total: | 67 | 6.2 | 0.085 | Total: | 9.4 | 0.09 |
| | | 10 dtex/50% Melting | | 0.0018 | | | | 0.12 | | |
| | | fiber Bicomponent PE | | | | | | | | |
| | | (sheath)/PET (core) | | | | | | | | |
| | | 5.8 dtex/20% Matrix | | | | | | | | |
| | | fiber PET 1.7 dtex | | | | | | | | |
| | Underside | 100% Absorbent fiber | 0.0005 | | | 1.7 | 0.035 | | | |
| | | CV 1.7 dtex | | | | | | | | |
| Sample 2 | Topside | 30% Matrix fiber PET | 0.0063 | Total: | 18 | 6.2 | 0.085 | Total: | 0.42 | 0.45 |
| | | 10 dtex/50% Melting | | 0.0068 | | | | 0.12 | | |
| | | fiber Bicomponent PE | | | | | | | | |
| | | (sheath)/PET (core) | | | | | | | | |
| | | 5.8 dtex/20% Matrix | | | | | | | | |
| | | fiber PET 1.7 dtex | | | | | | | | |
| | Underside | 70% Absorbent fiber | 0.0005 | | | 1.7 | 0.035 | | | |
| | | CV 1.7 dtex/30% | | | | | | | | |
| | | Melting fiber | | | | | | | | |
| | | Bicomponent PE | | | | | | | | |
| | | (sheath)/PP (core) | | | | | | | | |
| | | 1.7 dtex | | | | | | | | |
| Sample 3 | Topside | 60% Melting fiber | 0.0057 | Total: | 29 | 4.7 | 0.12 | Total: | 0.46 | 0.68 |
| | | Bicomponent PE | | 0.0062 | | | | 0.18 | | |
| | | (sheath)/PP (core) | | | | | | | | |
| | | 6.7 dtex/40% Melting | | | | | | | | |
| | | fiber bicomponent PE | | | | | | | | |
| | | (sheath)/PP (core) | | | | | | | | |
| | | 1.7 dtex | | | | | | | | |
| | Underside | 70% Absorbent fiber | 0.0005 | | | 1.7 | 0.06 | | | |
| | | CV 1.7 dtex/30% | | | | | | | | |
| | | Melting fiber | | | | | | | | |
| | | Bicomponent PE | | | | | | | | |
| | | (sheath)/PP (core) | | | | | | | | |
| | | 1.7 dtex | | | | | | | | |
| Sample 4 | Topside | 60% Melting fiber | 0.0055 | Total: | 24 | 4.7 | 0.1 | Total: | 0.8 | 0.07 |
| | | Bicomponent PE | | 0.0059 | | | | 0.14 | | |
| | | (sheath)/PP (core) | | | | | | | | |
| | | 6.7 dtex/40% Melting | | | | | | | | |
| | | fiber Bicomponent PE | | | | | | | | |
| | | (sheath)/PP (core) | | | | | | | | |
| | | 1.7 dtex | | | | | | | | |
| | Underside | 80% Absorbent fiber | 0.0004 | | | 1.6 | 0.04 | | | |
| | | CV 1.7 dtex/20% | | | | | | | | |
| | | Melting fiber | | | | | | | | |
| | | Bicomponent PE | | | | | | | | |
| | | (sheath)/PP (core) | | | | | | | | |
| | | 1.3 dtex | | | | | | | | |
| Sample 5 | Topside | 100% Melting fiber | 0.0038 | Total: | 30 | 4.4 | 0.07 | Total: | 0.8 | 0.52 |
| | | Bicomponent PE | | 0.0043 | | | | 0.13 | | |
| | | (sheath)/PET (core) | | | | | | | | |
| | | 4.4 dtex | | | | | | | | |
| | Underside | 80% Absorbent fiber | 0.0005 | | | 1.6 | 0.06 | | | |
| | | CV 1.7 dtex/20% | | | | | | | | |
| | | Melting fiber | | | | | | | | |
| | | Bicomponent PE | | | | | | | | |
| | | (Core)/PP (Core) | | | | | | | | |
| | | 1.3 dtex | | | | | | | | |

We claim:

1. A multilayer liquid absorption and distribution nonwoven fabric for hygiene products, the nonwoven fabric comprising:
a topside facing a user in hygiene products and an underside facing the absorbent body in hygiene products, wherein:
a fiber blend of the nonwoven fabric forming the underside has a lower average fiber titer than a fiber blend of the nonwoven fabric forming the topside,
the topside consists of a non-woven fabric, and the fiber blend of the topside consists of 50 to 100% by weight of melting fibers and 50 to 0% by weight of matrix fibers,
the underside consists of a non-woven fabric, and the fiber blend of the underside consists of 50 to 80% by weight of absorbent fibers and from 50 to 20% by weight of melting fibers,
the topside is exclusively thermally fusion-bonded to the underside, and
the density of the nonwoven fabric is 35 kg/m³ or less.

2. The multilayer liquid absorption and distribution nonwoven fabric of claim 1, wherein an average fiber titer of the fiber blend of the nonwoven fabric forming the underside is at least 2 dtex less than an average fiber titer of the fiber blend of the nonwoven fabric forming the topside.

3. The multilayer liquid absorption and distribution nonwoven fabric of claim 1, wherein the topside is thermally fusion-bonded to the underside by means of a hot-air treatment or by means of a hot-air treatment and a downstream calender treatment.

4. The multilayer liquid absorption and distribution nonwoven fabric of claim 2, wherein the topside is thermally fusion-bonded to the underside by means of a hot-air treatment or by means of a hot-air treatment and a downstream calender treatment.

5. The multilayer liquid absorption and distribution nonwoven fabric of claim 1, wherein the thickness of the topside is at least 60% of the total thickness.

6. The multilayer liquid absorption and distribution nonwoven fabric of claim 2, wherein the thickness of the topside is at least 60% of the total thickness.

7. The multilayer liquid absorption and distribution nonwoven fabric of claim 3, wherein the thickness of the topside is at least 60% of the total thickness.

8. The multilayer liquid absorption and distribution nonwoven fabric of claim 1, wherein fusible components of the melting fibers in the topside and in the underside are made of the same polymers.

9. The multilayer liquid absorption and distribution nonwoven fabric of claim 2, wherein fusible components of the melting fibers in the topside and in the underside are made of the same polymers.

10. The multilayer liquid absorption and distribution nonwoven fabric of claim 3, wherein fusible components of the melting fibers in the topside and in the underside are made of the same polymers.

11. The multilayer liquid absorption and distribution nonwoven fabric of claim 1, wherein the density of the underside is at least twice as high as the density of the topside.

12. The multilayer liquid absorption and distribution nonwoven fabric of claim 2, wherein the density of the underside is at least twice as high as the density of the topside.
